(19) United States
(12) Patent Application Publication
(10) Pub. No.: US 2023/0201602 A1
(43) Pub. Date: Jun. 29, 2023

(54) ELECTRICAL STIMULATION METHOD AND SYSTEM FOR IMPEDANCE COMPENSATION

(71) Applicant: GIMER MEDICAL. Co. LTD., New Taipei City (TW)

(72) Inventors: Wan-Ting CHIANG, New Taipei City (TW); Jian-Hao PAN, New Taipei City (TW)

(73) Assignee: GIMER MEDICAL. Co. LTD., New Taipei City (TW)

(21) Appl. No.: 17/979,524

(22) Filed: Nov. 2, 2022

(30) Foreign Application Priority Data
Dec. 29, 2021 (CN) 202111633749.6

(51) Int. Cl. A61N 1/36; A61N 1/05

(52) U.S. Cl. CPC A61N 1/3614; A61N 1/0504; A61N 1/36175

(57) ABSTRACT

An electrical stimulation method for impedance compensation is provided. The electrical stimulation method for impedance compensation is applied to an electrical stimulation device for providing high frequency electrical stimulation. In the above method, by an impedance compensation device, a high-frequency environment is provided and a first impedance value of a lead is calculated according to at least one of a measured first resistance value, a measured first capacitance value and a measured first inductance value of the lead is calculated. By the impedance compensation device, the high-frequency environment is provided and a second impedance value of the electrical stimulation device is calculated according to at least one of a measured second resistance value, a measured second capacitance value and a measured second inductance value of the electrical stimulation device. The first impedance value and the second impedance value are stored for calculating a tissue impedance.

### CROSS REFERENCE TO RELATED APPLICATIONS

This application claims priority of China Patent Application No. 202111633749.6, filed on Dec. 29, 2021, the entirety of which is incorporated by reference herein.

### BACKGROUND

### Technology Field

The embodiments of present invention mainly relates to an electrical stimulation technique.

### Description of the Related Art

In recent years, several therapeutic electrical nerve stimulation devices have been developed, and tens of thousands of people receive surgery for the implantation of electrical stimulation devices each year. With the development of precision manufacturing techniques, medical instruments (such as electrical stimulation devices) have been miniaturized and may be implanted into the human body.

Conventionally, the impedance values of an electrical stimulation device and a lead are measured and written into firmware in the electrical stimulation device or an external control device before it leaves the factory. However, in some cases (for example, when the electrical stimulation device is under a high-frequency environment), the impedance values of the electrical stimulation device and the lead may change, causing a mismatch between the actual impedance value and the value stored in the firmware. This can result in inaccuracies in the parameters of the electrical stimulation signal that is generated, and may affect the device's curative effect.

### SUMMARY

In view of the issues of the prior arts described above, an embodiment of the present invention provides an electrical stimulation method and system for impedance compensation.

An embodiment of the present invention provides an electrical stimulation method for impedance compensation. The electrical stimulation method for impedance compensation can be applied to an electrical stimulation device that provides high-frequency electrical stimulation. The steps of the electrical stimulation method for impedance compensation include the following. An impedance compensation device provides a high-frequency environment and calculates a first impedance value of a lead using at least one of the measured first resistance value, the measured first capacitance value, or the measured first inductance value of the lead. The impedance compensation device provides a high-frequency environment and calculates a second impedance value of the electrical stimulation device using at least one of the measured second resistance value, the measured second capacitance value, and the measured second inductance value of the electrical stimulation device. The first impedance value and the second impedance value are stored, for later use in calculating the compensation for a tissue impedance value.

An embodiment of the present invention provides an electrical stimulation system. The electrical stimulation system can be applied in a high-frequency electrical stimulation operation. The electrical stimulation system includes a lead, an electrical stimulation device, and an impedance compensation device. The impedance compensation device provides a high-frequency environment. The impedance compensation device calculates a first impedance value of the lead using at least one of the measured first resistance value, the measured first capacitance value, or the measured first inductance value of the lead. The impedance compensation device calculates a second impedance value of the electrical stimulation device using at least one of the measured second resistance value, the measured second capacitance value, and the measured second inductance value of the electrical stimulation device. The first impedance value and the second impedance value are stored in the electrical stimulation device for calculating the compensation for a tissue impedance value.

An embodiment of the present invention provides an electrical stimulation method for impedance compensation. The electrical stimulation method for impedance compensation can be employed by an electrical stimulation device that provides a high-frequency electrical stimulation. The steps of the electrical stimulation method for impedance compensation are as follows. The impedance compensation device provides a high-frequency environment. The impedance compensation device calculates an impedance value of the electrical stimulation device using at least one of the measured resistance value, the measured capacitance value, and the measured inductance value of the electrical stimulation device. The impedance compensation device stores the impedance value so that it can be used to calculate the compensation for a tissue impedance value.

An embodiment of the present invention provides an electrical stimulation system. The electrical stimulation system uses a high-frequency electrical stimulation operation. The electrical stimulation system includes an electrical stimulation device and an impedance compensation device. The impedance compensation device provides a high-frequency environment. The impedance compensation device calculates an impedance value of the electrical stimulation device using at least one of the measured resistance value, the measured capacitance value, and the measured inductance value of the electrical stimulation device. The impedance value is stored in the electrical stimulation device for calculating the compensation for a tissue impedance value.

Persons skilled in the art may achieve additional features and advantages of the present invention by modifying and altering the electrical stimulation method for impedance compensation and the electrical stimulation system disclosed in the detailed description of the present disclosure without departing from the spirit and scope of the present invention.

### BRIEF DESCRIPTION OF THE DRAWINGS

FIG. 1 shows a block diagram of an electrical stimulation device 100 according to an embodiment of the present invention;

FIG. 2A shows a schematic diagram of the electrical stimulation device 100 according to an embodiment of the present invention;

FIG. 2B shows a schematic diagram of the electrical stimulation device 100 according to another embodiment of the present invention;

FIG. 3 shows a waveform diagram of an electrical stimulation signal of the electrical stimulation device according to an embodiment of the present invention;

FIG. 4 shows a detailed schematic diagram of the electrical stimulation device 100 according to an embodiment of the present invention;

FIG. 5A shows a first set of predetermined target energy values according to an embodiment of the present invention;

FIG. 5B shows a second set of predetermined target energy values according to another embodiment of the present invention;

FIG. 6 shows a block diagram of an impedance compensation device 600 according to an embodiment of the present invention;

FIG. 7A shows a schematic diagram of an impedance compensation model according to an embodiment of the present invention;

FIG. 7B shows a schematic diagram of an impedance compensation model according to another embodiment of the present invention;

FIG. 8 shows a flow diagram 800 of an electrical stimulation method for impedance compensation according to an embodiment of the present invention; and

FIG. 9 shows a flow diagram 900 of an electrical stimulation method for impedance compensation according to another embodiment of the present invention.

### DETAILED DESCRIPTION OF THE INVENTION

The following description explains preferred implementations of the present invention, which is made for the purpose of illustrating the general principles of the invention and should not be taken in a limiting sense. The scope of the invention is best determined by reference to the appended claims.

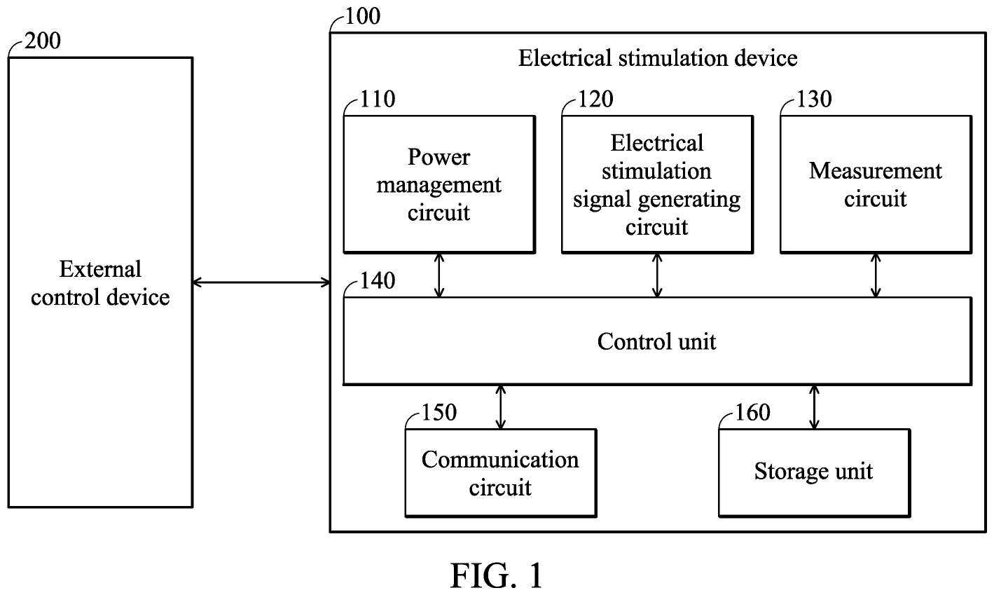

FIG. 1 shows a block diagram of an electrical stimulation device 100 according to an embodiment of the present invention. As shown in FIG. 1, the electrical stimulation device 100 may include at least a power management circuit 110, an electrical stimulation signal generating circuit 120, a measurement circuit 130, a control unit 140, a communication circuit 150 and a storage unit 160. Note that the block diagram shown in FIG. 1 is merely illustrated for the convenience of explaining the embodiments of the present invention, but the invention is not limited thereto. The electrical stimulation device 100 may include other elements.

According to an embodiment of the present invention, the electrical stimulation device 100 may be electrically coupled to an external control device 200. The external control device 200 may have an operation interface. According to the input by a user via the operation interface, the external control device 200 may generate a command or signal to send to the electrical stimulation device 100, and it may then send that command or signal to the electrical stimulation device 100 via wired communication (e.g. a transmission line).

Additionally, according to another embodiment of the present invention, the external control device 200 may send the command or signal to the electrical stimulation device 100 by wireless communication, such as Bluetooth, Wi-Fi or near field communication (NFC).

According to an embodiment of the present invention, the electrical stimulation device 100 may be an implantable electrical stimulation device, an external electrical stimulation device with a lead implanted into a human body, or a transcutaneous electrical nerve stimulation device (TENS). According to an embodiment of the present invention, when the electrical stimulation device 100 is a non-implantable electrical stimulation device (e.g. an external electrical stimulation device or a transcutaneous electrical-stimulation device), the electrical stimulation device 100 and the external control device 200 may be integrated as a single device. According to an embodiment of the present invention, the electrical stimulation device 100 may be an electrical stimulation device with a battery, or an electrical stimulation device with power wirelessly transmitted from the external control device 200. According to an embodiment of the present invention, in a trial phase, the electrical stimulation device 100 is an external electrical stimulation device with a lead implanted into a human body, the lead has electrodes thereon, and the external electrical stimulation device sends an electrical stimulation signal to a corresponding target region through the electrodes on the lead. In the trial phase, after the end of the lead with the electrode is implanted into the human body, the other end of the lead is connected with the external control device 200, and the external electrical stimulation device may send an electrical stimulation signal to evaluate whether the treatment is effective, and to check whether the lead functions properly and whether the position of lead implantation is correct. In the trial phase, the external control device 200 first pairs wirelessly with the external electrical stimulation device (i.e. the non-implantable electrical stimulation device). After the lead is implanted into the human body, the external electrical stimulation device (i.e. the non-implantable electrical stimulation device) is connected to the lead, and the external control device 200 wirelessly controls the external electrical stimulation device (i.e. the non-implantable electrical stimulation device) to perform an electrical stimulation of the human body. According to an embodiment of the present invention, if the treatment is evaluated as effective in the trial phase, a permanent implantation phase may be performed. In the permanent implantation phase, the electrical stimulation device 100 may be implanted into the human body along with the lead, and the electrical stimulation device 100 sends the electrical stimulation signal to the corresponding target region through the electrodes on the lead. When the external control device 200 is entering the permanent implantation phase, the user or a medical doctor first needs to tap a phase change card to the external control device 200 to change the using status of the external control device 200 from the trial phase to the permanent implantation phase through NFC, and the external control device 200 may select an upper limit target energy value and a lower limit target energy value from a first set of target energy values according to a given electrical stimulation level. Subsequently, the external control device 200 may generate a second set of target energy values according to the upper limit target energy value and the lower limit target energy value (which is further described below). In addition, before the permanent implantation surgery or in the permanent implantation phase, the external control device 200 and the implantable electrical stimulation device are wirelessly paired, the external electrical stimulation device (i.e. the non-implantable electrical stimulation device) is removed, and the electrical stimulation device 100 (i.e. the implantable electrical stimulation device) is connected with the lead and implanted into the human body.

According to an embodiment of the present invention, the power management circuit 110 is configured to provide power to the elements and circuits within the electrical stimulation device 100. The power provided by the power management circuit 110 may come from a built-in rechargeable battery or the external control device 200, but the invention is not limited thereto. The external control device 200 may provide power to the power management circuit 110 by a wireless power-providing technique. The power management circuit 110 may be enabled or disabled according to a command from the external control device 200. According to an embodiment of the present invention, the power management circuit 110 may include a switch circuit (not shown in the figure). The switch circuit may be turned on or off according to the command from the external control device 200, so as to enable or disable the power management circuit 110.

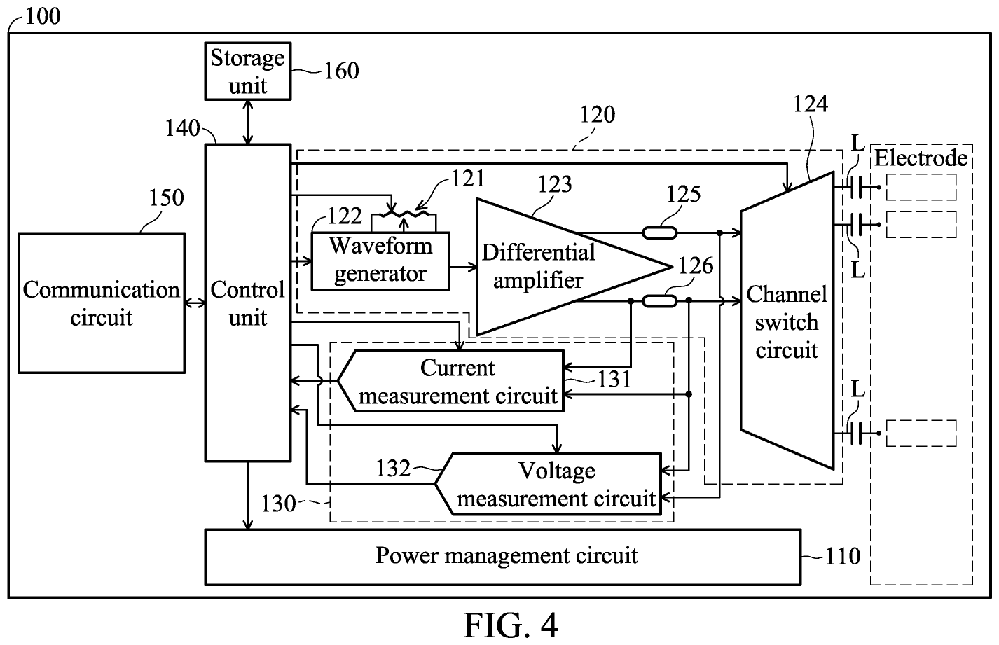

According to an embodiment of the present invention, the electrical stimulation signal generating circuit 120 is configured to generate an electrical stimulation signal. The electrical stimulation device 100 may send the electrical stimulation signal to the electrode on the lead through at least one lead, so as to perform an electrical stimulation of a target region in the body of a user (a human or an animal) or a patient. Examples of possible target regions include a spinal cord, a spinal nerve, a dorsal root ganglia (DRG), a cranial nerve, a vagus nerve, a trigeminal nerve, a lateral recess, and a peripheral nerve, but the invention is not limited thereto. The structure of the electrical stimulation generating circuit 120 is explained in more detail in FIG. 4.

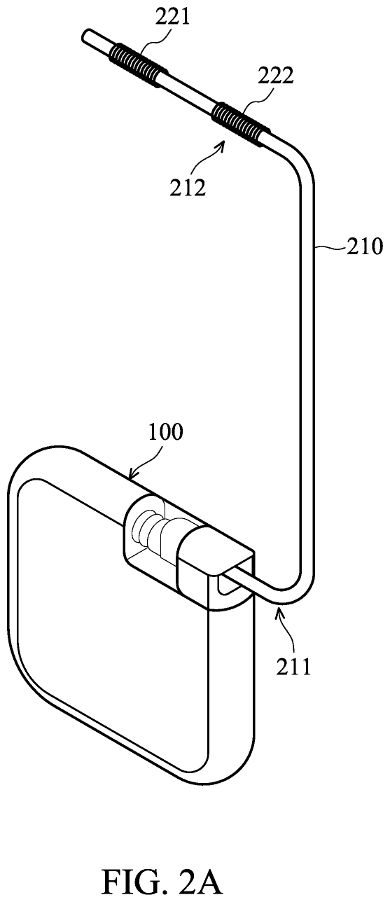

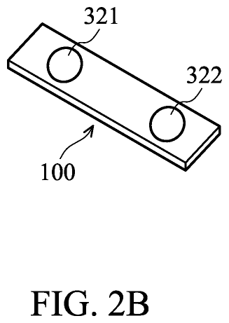

FIG. 2A shows a schematic diagram of the electrical stimulation device 100 according to an embodiment of the present invention. As shown in FIG. 2A, an electrical stimulation signal may be output to a lead 210 to allow the electrical stimulation signal to be sent from an end 211 of the lead 210 to another end 212 of the lead 210 (an electrode 221 or 222), so as to perform an electrical stimulation operation in a target region. In an embodiment of the present invention, the electrical stimulation device 100 and the lead 210 are electrically connected in a detachable manner, but the invention is not limited thereto. For example, the electrical stimulation device 100 and the lead 210 may be an integrally formed device.

FIG. 2B shows a schematic diagram of the electrical stimulation device 100 according to another embodiment of the present invention. As shown in FIG. 2B, electrodes 321 and 322 may be directly formed on a side of the electrical stimulation device 100. The electrical stimulation signal may be transmitted to the electrode 321 or the electrode 322 for performing an electrical stimulation operation at a target region. That is, in this embodiment, the electrical stimulation device 100 does not need a lead to send the electrical stimulation signal to the electrodes 321 and 322.

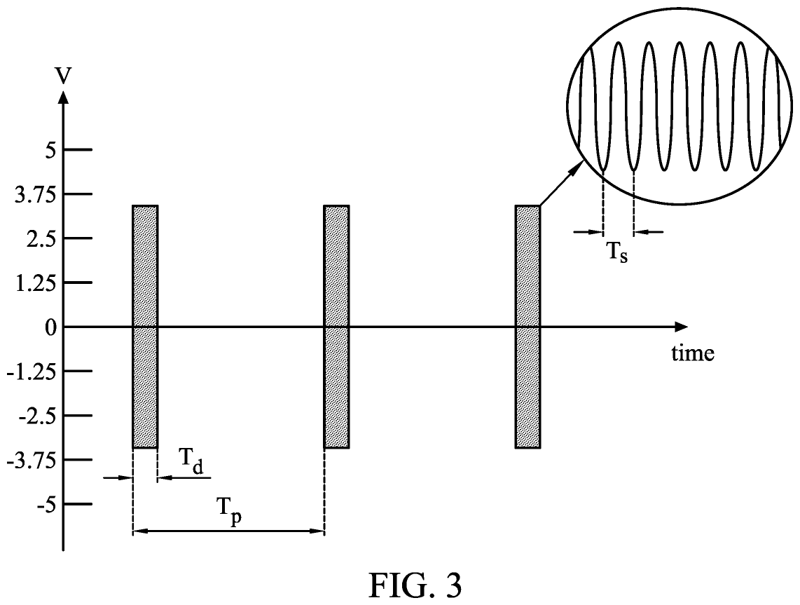

FIG. 3 shows a waveform diagram of an electrical stimulation signal of the electrical stimulation device according to an embodiment of the present invention. As shown in FIG. 3, according to an embodiment of the present invention, the electrical stimulation signal may be a pulsed radio-frequency (PRF) signal (also referred to as a pulsed signal), a continuous sine wave, a continuous triangular form or the like, but the present invention is not limited thereto. Additionally, when the electrical stimulation signal is a pulsed alternating signal, a pulse cycle time Tₚ includes a pulsed signal and at least one resting period, and the pulse cycle time Tₚ is the reciprocal of a pulse repetition frequency. The range of the pulse repetition frequency (also referred to as the pulse frequency range) is, for example, between 0 and 1 KHz, preferably between 1 and 100 Hz, and the pulse repetition frequency of the electrical stimulation signal in the present embodiment is, for example, 2 Hz. Furthermore, a duration time T_{d} (i.e. a pulse width) of a pulse in a pulse cycle time is between, for example, 1 and 250 ms (milliseconds), preferably between 10 and 100 ms, and the duration time T_{d} of the present embodiment is described as 25 ms as an example. In the present embodiment, the frequency of the electrical stimulation signal is 500 KHz. In other words, the cycle time Tₛ of the electrical stimulation signal is approximately 2 microseconds (μs). Additionally, the frequency of the electrical stimulation signal is the intra-pulse frequency within each pulsed alternating signal shown in FIG. 3. In some embodiments, the range of the intra-pulse frequency of the electrical stimulation signal is between, for example, 1 KHz and 1000 KHz. Note that in the embodiments of the present invention, if merely a “frequency” of the electrical stimulation signal is described, it refers to the intra-pulse frequency of the electrical stimulation frequency. Furthermore, the range of the intra-pulse frequency of the electrical stimulation frequency is, for example, between 200 KHz and 800 KHz. Moreover, the range of the intra-pulse frequency of the electrical stimulation frequency is, for example, between 480 KHz and 520 KHz. Furthermore, the intra-pulse frequency of the electrical stimulation frequency is, for example, 500 KHz. The voltage range of the electrical stimulation signal may be between −25V and +25V. Furthermore, the voltage range of the electrical stimulation signal may be further between −20V and +20V. The current range of the electrical stimulation signal may be between 0 and 60 mA. Furthermore, the current range of the electrical stimulation signal may be further between 0 and 50 mA.

According to an embodiment of the present invention, the user may operate the electrical stimulation device 100 to perform an electrical stimulation when needed (for example, when a symptom worsens or not relieved). After the electrical stimulation device 100 performs an electrical stimulation to the target region, the electrical stimulation device 100 needs to wait for a limit time before performing the next electrical stimulation to the target region. For example, after the electrical stimulation device 100 performs an electrical stimulation, the electrical stimulation device 100 needs to wait for 30 minutes (i.e. the limit time) before performing the next electrical stimulation to the target region, but the present invention is not limited thereto. The limit time may be any time duration within 45 minutes, 1 hour, 4 hours or 24 hours.

According to an embodiment of the present invention, the measurement circuit 130 may measure a voltage value and a current value of the electrical stimulation signal according to the electrical stimulation generated by the electrical stimulation signal generating circuit 120. Additionally, the measurement circuit 130 may measure a voltage value and a current value of a tissue in a target region in the body of the user or patient. According to an embodiment of the present invention, the measurement circuit 130 may adjust the current and the voltage of the electrical stimulation signal according to an instruction from the control unit 140. The structure of the measurement circuit 130 is explained in more detail below with reference to FIG. 4.

According to an embodiment of the present invention, the control unit 140 may be a controller, a microcontroller or a processor, but the present invention is not limited thereto. The control unit 140 may be configured to control the electrical stimulation signal generating circuit 120 and the measurement circuit 130. The operation of the control unit 140 is explained below referring to FIG. 4.

According to an embodiment of the present invention, the communication circuit 150 may be configured to communicate with the external control device 200. The communication circuit 150 may send a command or signal received from the external control device 200 to the control unit 140, and send data measured by the electrical stimulation device 100 to the external control device 200. According to an embodiment of the present invention, the communication circuit 150 may communicate with the external control device 200 via wireless or wired communication.

According to an embodiment of the present invention, when performing electrical stimulation, all the electrodes of the electrical stimulation device 100 are activated (or enabled). Thus, the user does not need to select which of the electrodes on the lead are to be activated, and does not need to select which of the activated electrodes are negative or positive in polarities. For example, if the electrical stimulation device 100 is equipped with eight electrodes, then the eight electrodes may be four positive electrodes and four negative electrodes arranged alternately.

Compared to a conventional electrical stimulation, which is a low-frequency (e.g. 10 KHz) pulsed signal prone to cause a tingling pain or paresthesia of the user, resulting in a discomfort of the user, in an embodiment of the present invention, the electrical stimulation is a high-frequency (e.g. 500 KHz) pulsed signal, which causes no or little paresthesia of the user.

According to an embodiment of the present invention, the storage unit 160 may be a volatile memory (e.g. a random access memory (RAM)), a non-volatile memory (e.g. a flash memory), a read-only memory (ROM), a hard disk drive, or a combination thereof. The storage unit 160 may be configured to store a file and data required for performing an electrical stimulation. According to an embodiment of the present invention, the storage unit 160 may be configured to store information related to a look-up table provided by the external control device.

FIG. 4 shows a schematic diagram of the electrical stimulation device 100 according to an embodiment of the present invention. As shown in FIG. 4, the electrical stimulation signal generating circuit 120 may include a variable resistor 121, a waveform generator 122, a differential amplifier 123, a channel switch circuit 124, a first resistor 125 and a second resistor 126. The measurement circuit 130 may include a current measurement circuit 131 and a voltage measurement circuit 132. Note that the schematic diagram shown in FIG. 4 is illustrated merely for the convenience of explaining an embodiment of the present invention, but the present invention is not limited thereto. The electrical stimulation device 100 may include additional elements, or include additional equivalent circuits.

As shown in FIG. 4, according to an embodiment of the present invention, the variable resistor 121 may be coupled to a serial peripheral interface (SPI) (not shown in the figure) of the control unit 140. The control unit 140 may send a command to the variable resistor 121 through the SPI to adjust the resistance of the variable resistor 121, so as to adjust the magnitude of the electrical stimulation signal to output. The waveform generator 122 may be coupled to a pulse width modulation (PWM) signal generator (not shown in the figure) of the control unit 140. The PWM signal generator may generate a square wave and send the square wave to the waveform generator 122. After receiving the square wave generated by the PWM signal generator, the waveform generator 122 transforms the square wave to a sine wave signal and sends the sine wave signal to the differential amplifier 123. The differential amplifier 123 may transform the sine wave signal to a differential signal (i.e. the electrical stimulation signal to output) and sends the differential signal to the channel switch circuit 124 through the first resistor 125 and the second resistor 126. The channel switch circuit 124 may sequentially send the differential signal (i.e. the electrical stimulation signal to output) to the electrode corresponding to each channel through a lead L according to the command from the control unit 140.

As shown in FIG. 4, according to an embodiment of the present invention, the current measurement circuit 131 and the voltage measurement circuit 132 may be coupled to the differential amplifier 123 to obtain a current value and a voltage value of the differential signal (i.e. the electrical stimulation signal to output). Additionally, the current measurement circuit 131 and the voltage measurement circuit 132 may be configured to measure a voltage value and a current value of a tissue in a target region in the body of a user or patient. In addition, the current measurement circuit 131 and the voltage measurement circuit 132 may be coupled to an input/output (I/O) interface of the control unit (not shown in the figure) to receive a command from the control unit 140. According to the command from the control unit 140, the current measurement circuit 131 and the voltage measurement circuit 132 may adjust the current and the voltage of the electrical stimulation signal to a current value and a voltage value suitable for the control unit 140 to process. For example, if the voltage value measured by the voltage measurement circuit 132 is ±10V and the voltage value suitable for the control unit 140 to process is 0 to 3V, then the voltage measurement circuit 132 may scale down the voltage value to ±1.5V and then shift the voltage value to 0-3V according to the command from the control unit 140.

After the current measurement circuit 131 and the voltage measurement circuit 132 adjust the current value and the voltage value, the current measurement circuit 131 and the voltage measurement circuit 132 send the adjusted electrical stimulation signal to an analog-to-digital converter (ADC) (not shown in the figure) of the control unit 140. The ADC samples the electrical stimulation signal for the control unit 140 to perform subsequent computation and analysis.

According to an embodiment of the present invention, when an electrical stimulation in a target region in the body of a patient is desired, a user (may be medical personnel or the patient himself/herself) may select an electrical stimulation level from multiple electrical stimulation levels on an operation interface of the external control device 200. In an embodiment of the present invention, different electrical stimulation levels may correspond to different target energy values. The target energy values may be a set of predetermined energy values. When the user selects an electrical stimulation level, the electrical stimulation device 100 may be informed of the energy value (in millijoules) to provide to the target region to perform the electrical stimulation according to the target energy value corresponding to the electrical stimulation level selected by the medical doctor or the user. According to an embodiment of the present invention, in the trial phase, a plurality of target energy value corresponding to a plurality of electrical stimulation levels may be regarded as a first set of predetermined target energy values. According to an embodiment of the present invention, the first set of predetermined target energy values (i.e. the plurality of target energy values) may be a linear sequence, an arithmetic sequence or a geometric sequence, but the present invention is not limited thereto.

According to an embodiment of the present invention, in the trial phase, the external control device 200 may have a first look-up table. In this embodiment, each electrical stimulation level and its corresponding target energy value may be recorded in the first look-up table. Thus, according to the electrical stimulation level selected by the user, the external control device 200 may search the first look-up table and obtain the target energy value corresponding to the electrical stimulation level selected by the user from the first set of target energy values. After obtaining the target energy value corresponding to the electrical stimulation level selected by the user, the external control device 200 sends the target energy value to the electrical stimulation device 100. Then the electrical stimulation device 100 may perform the electrical stimulation to the target region according to the target energy value.

According to another embodiment of the present invention, the electrical stimulation device 100 may have a built-in first look-up table (e.g. a first look-up table stored in in the storage unit 160). In this embodiment, each electrical stimulation level and its corresponding target energy value may be recorded in the first look-up table. When the user selects an electrical stimulation level from the external control device 200, the external control device 200 sends a command to inform the control unit 140 of the electrical stimulation device 100 of the electrical stimulation level selected by the user. Then, the control unit 140 may select the target energy value corresponding to the electrical stimulation level selected by the user from the first set of target energy values according to the built-in first look-up table. After obtaining the target energy value, the electrical stimulation device 100 may perform the electrical stimulation to the target region according to the selected target energy value until the corresponding first target energy value is sent to the target region, then stop the electrical stimulation to finish an electrical stimulation treatment.

According to another embodiment of the present invention, the communication circuit 150 may first obtain the electrical stimulation level selected by the user and the first look-up table from the external control device 200. In this embodiment, each electrical stimulation level and its corresponding target energy value may be recorded in the first look-up table. Then, the control unit 140 selects the target energy value corresponding to the electrical stimulation level selected by the user from the first set of target energy values according to the electrical stimulation level selected by the user and the first look-up table obtained from the external control device 200. After obtaining the target energy value, the electrical stimulation device 100 may perform the electrical stimulation to the target region according to the selected target energy value.

According to an embodiment of the present invention, the user may start from selecting the lowest electrical stimulation level (corresponding to the smallest target energy value in the first set of target energy values) and select the next target energy value in the first set of target energy values after finishing the electrical stimulation and the limit time elapsed. When the user finds a preferred or effective target energy value in electrical stimulation, such target energy value may be regarded as a given target energy value, and the electrical stimulation level corresponding to the given target energy value may be regarded as a given electrical stimulation level.

According to an embodiment of the present invention, in the permanent implantation phase, the external control device 200 (e.g. a controller of the external control device 200) may select an upper limit target energy value and a lower limit target energy value from the first set of target energy values according to the given electrical stimulation level. Then, the external control device 200 (e.g. a controller of the external control device 200) may generate a second set of target energy values according to the upper limit target energy value and the lower limit target energy value. In this embodiment, the external control device 200 (e.g. a controller of the external control device 200) generates a second look-up table according to the electrical stimulation level corresponding to each target energy value in the second set of target energy values. The external control device 200 sends the second look-up table or related parameter information to the electrical stimulation device 100. When the user operates the external control device 200, the electrical stimulation device 100 performs the electrical stimulation operation according to the second look-up table or the related parameter information. According to an embodiment of the present invention, in the trial phase, the electrical stimulation is performed to human body by an external electrical stimulation device (i.e. a non-implantable electrical stimulation device) according to a first set of target energy values selected by the user in the first look-up table; in the permanent implantation phase, the electrical stimulation is performed to human body by the electrical stimulation device 100 (i.e. an implantable electrical stimulation device) according to a second set of target energy values selected by the user in the second look-up table. In an embodiment of the present invention, the electrical stimulation device 100 performs the electrical stimulation to the target region until the corresponding second target energy value is sent to the target region, then stop the electrical stimulation to finish an electrical stimulation treatment.

According to another embodiment of the present invention, in the permanent implantation phase, the electrical stimulation device 100 may select an upper limit target energy value and a lower limit target energy value from the first set of target energy values according to the given electrical stimulation level. Then, the electrical stimulation device 100 may generate a second set of target energy values according to the upper limit target energy value and the lower limit target energy value. In this embodiment, the electrical stimulation device 100 generates a second look-up table according to the electrical stimulation level corresponding to each target energy value in the second set of target energy values. The electrical stimulation device 100 sends the second look-up table or related parameter information to the external control device 200. When the user operates the external control device 200, the electrical stimulation device 100 performs the electrical stimulation operation according to the second look-up table or the related parameter information.

According to an embodiment of the present invention, the second set of target energy values may be a linear sequence, an arithmetic sequence or a geometric sequence, but the present invention is not limited thereto. According to an embodiment of the present invention, the number of target energy values included in the first set of target energy values may be equal to the number of target energy values included in the second set of target energy values. According to another embodiment of the present invention, the number of target energy values included in the first set of target energy values may be different from the number of target energy values included in the second set of target energy values.

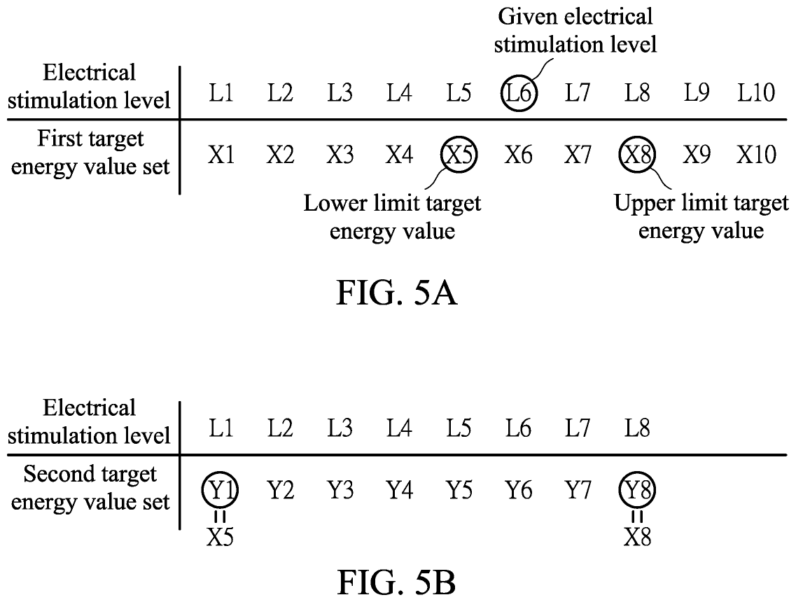

FIG. 5A shows a first set of target energy values according to an embodiment of the present invention. FIG. 5B shows a second set of target energy values according to an embodiment of the present invention. Note that FIGS. 5A and 5B are illustrated merely to explain an embodiment of the present invention, but the present invention is not limited to the first set of target energy values and the second set of target energy values shown in FIGS. 5A and 5B.

As shown in FIG. 5A, the corresponding relations of the various electrical stimulation levels and the various first target energy are stored in the first look-up table, wherein the first set of target energy value may include target energy values X1 to X10. The electrical stimulation levels Level 1 (L1) to Level 10 (L10) correspond to target energy values X1 to X10 respectively, and the target energy values are energy values measured in millijoules. In addition to corresponding to the target energy values, the electrical stimulation levels L1 to L10 may correspond to different current values or voltage values. In this embodiment, in the trial phase, when the given electrical stimulation level selected by the user is L6 (i.e. the corresponding given target energy value is X6), the predetermined upper limit target energy value is X8 and the lower limit target energy value is X5. The upper limit target energy value X8 and the given target energy value X6 are separated by one target energy value, and the lower limit target energy value X5 and the given target energy value X6 are not separated by any target energy value.

In the permanent implantation phase, after obtaining the upper limit target energy value X8 and the lower limit target energy value X5, the electrical stimulation device 100 or the external control device 200 may generate a second set of target energy values according to the upper limit target energy value X8 and the lower limit target energy value X5. As shown in FIG. 5B, the second set of target energy values may include target energy values Y1 to Y8, and the target energy values Y1 to Y8 correspond to the electrical stimulation levels L1 to L8 of the external control device 200 respectively. In addition, in this embodiment, the minimum target energy value Y1 of the second set of target energy values corresponds to the lower limit target energy value X5, namely Y1 is equal to X5, and the maximum target energy value Y8 corresponds to the upper limit target energy value X8, namely Y8 is equal to X8. In the permanent implantation phase, the electrical stimulation device 100 and the external control device 200 may perform the electrical stimulation operation according to the second set of target energy values.

According to an embodiment of the present invention, when corresponding to a given electrical stimulation level in the trial phase, the first target energy values include an upper limit target energy value and a lower limit target energy value. The upper limit target energy value and the lower limit target energy value are brought into the permanent implantation phase, the upper limit target energy value is the greatest target energy value in the second set of target energy values, and the lower limit target energy value is the smallest target energy value in the second set of target energy values (as shown in FIG. 5B). As such, it may be ensured that the electrical stimulation in the permanent implantation phase may be performed to the user at an energy intensity near the selected given electrical stimulation level, increasing safety.

According to an embodiment of the present invention, the upper limit target energy value and the given target energy value are separated by a first number of target energy values, and the lower limit target energy value and the given target energy value are separated by a second number of target energy values. According to an embodiment of the present invention, the first number (e.g. two) is larger than the second number (e.g. one) (as shown in FIG. 5A). According to another embodiment of the present invention, the first number may be equal to the second number.

According to an embodiment of the present invention, the given target energy value is not included in the second set of target energy values (as shown in FIG. 5B). According to another embodiment of the present invention, the given target energy value may be included in the second set of target energy values.

According to an embodiment of the present invention, the trial phase and the permanent implantation phase may be respectively separated into a non-electrical stimulation phase and an electrical stimulation phase. That is, the trial phase includes a non-electrical stimulation phase and an electrical stimulation phase, and the permanent implantation phase includes a non-electrical stimulation phase and an electrical stimulation phase as well. The non-electrical stimulation phase is the synchronization process when the electrical stimulation device 100 and the external control device 200 are just started and connected, or after the connection of the electrical stimulation device 100 and the external control device 200 and before the user starts the electrical stimulation. The electrical stimulation phase is the phase in which the electrical stimulation device has started to provide the electrical stimulation treatment. Note that the methods described herein for calculating the tissue impedance value are equally applicable to both the trial phase and the permanent implantation phase.

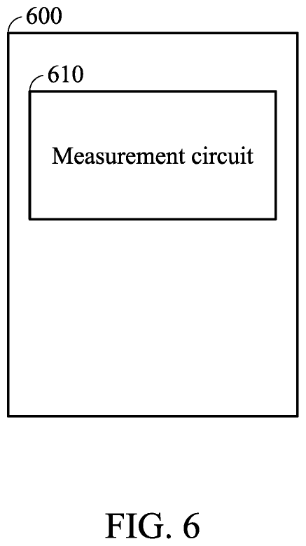

FIG. 6 shows a block diagram of an impedance compensation device 600 according to an embodiment of the present invention. As shown in FIG. 6, the impedance compensation device 600 may include a measurement circuit 610, but the present invention is not limited thereto. The measurement circuit 610 may be configured to measure an impedance value Z_{Inner} of the electrical stimulation device 100 and am impedance value Z_{Lead} of the lead. According to an embodiment of the present invention, the impedance compensation device 600 (or the measurement circuit 610) may include a related circuit structure shown in FIG. 4.

According to an embodiment of the present invention, when the measurement circuit 610 is measuring the electrical stimulation device 100 shown in FIG. 2A, the measurement circuit 610 first provides a high-frequency environment, wherein the frequency (e.g. 500 KHz) equals to the frequency of the electrical stimulation signal performing the electrical stimulation at the target region. Then, the measurement circuit 610 measures a resistance value R_{Lead}, a capacitance value C_{Lead} and an inductance value L_{Lead} of the lead, and calculates the impedance of the lead Z_{Lead} under a high-frequency signal according to at least one of the measured resistance value R_{Lead}, the capacitance value C_{Lead}, and inductance value L_{Lead} In addition, the measurement circuit 610 measures the resistance value R_{Inner}, the capacitance value C_{Inner}, and the inductance value Lᵢₙₙₑᵣ of the electrical stimulation device 100, and calculates the impedance of the electrical stimulation device 100 Z_{Inner} according to at least one of the measured resistance value R_{Inner}, capacitance value C_{Inner} and inductance value L_{Inner}. In an embodiment of the present invention, the measurement of the inductance Lᵢₙₙₑᵣ of the electrical stimulation device 100 is not required. The measurement circuit 610 writes the calculated impedance value of the lead Z_{Lead} and the impedance value of the electrical stimulation device 100 Z_{Inner} to the firmware of the electrical stimulation device 100.

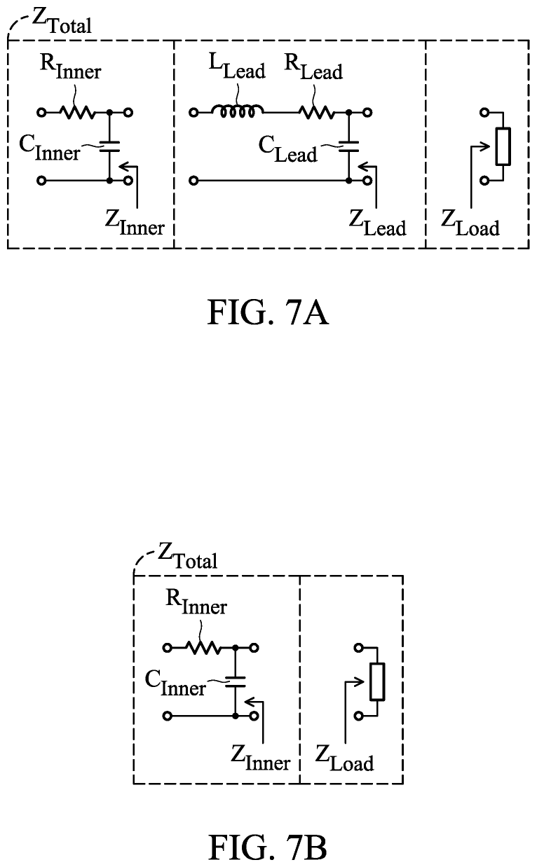

When the electrical stimulation device 100 is calculating the tissue impedance value Z_{Load} at the target region, the electrical stimulation device 100 may subtract the impedance value of the lead Z_{Lead} and the impedance value of the electrical stimulation device 100 Z_{Inner} from the calculated total impedance value Z_{Total} to obtain the tissue impedance Z_{Load} at the target region. As shown in the impedance compensation illustrated in FIG. 7A, Z_{Load}=Z_{Total}−Z_{Inner}−Z_{Lead}, but the present invention is not limited thereto. In an embodiment of the present invention, the total impedance value Z_{Total} may be calculated by a calculation module 144 through the current measured by the current measurement circuit 131 and the voltage measured by the voltage measurement circuit 132 (i.e. R=V/I). As the calculation of the impedance value Z_{Lead} of the lead and the impedance value Z_{Inner} of the electrical stimulation device 100 may refer to the formula Z=R+j(XL−XC), wherein R is the resistance, XL is the inductive reactance and XC is the capacitive reactance, such calculation is well-known by persons skilled in the art and is thus not described in detail herein.

According to another embodiment of the present invention, when the measurement circuit 610 is measuring the electrical stimulation device 100 shown in FIG. 2B, the measurement circuit 610 first provides a high-frequency environment. The measurement circuit 610 measures a resistance value R_{Inner}, a capacitance value C_{Inner} and an inductance value L_{Inner} of the electrical stimulation device 100, and calculates the impedance of the electrical stimulation device 100 Z_{Inner} according to at least one of the measured resistance value R_{Inner}, capacitance value C_{Inner}, or inductance value L_{Inner}. In an embodiment of the present invention, the measurement of the inductance Lᵢₙₙₑᵣ of the electrical stimulation device 100 is not required. The measurement circuit 610 writes the calculated impedance value of the electrical stimulation device 100 Z_{Inner} to the firmware of the electrical stimulation device 100. When the electrical stimulation device 100 is calculating the tissue impedance value Z_{Load} at the target region, the electrical stimulation device 100 may subtract the impedance value of the electrical stimulation device 100 Z_{Inner} from the calculated total impedance value Z_{Total} to obtain the tissue impedance Z_{Load} at the target region. As shown in the impedance compensation illustrated in FIG. 7B, Z_{Load}=Z_{Total}−Z_{Inner}, but the present invention is not limited thereto.

According to an embodiment of the present invention, the measurement circuit 610 may simulate a high-frequency environment according to an electrical stimulation frequency used by the electrical stimulation device 100. According to an embodiment of the present invention, the range of the pulse frequency of the high-frequency environment provided by the measurement circuit 610 may be between 1 KHz and 1000 KHz. According to an embodiment of the present invention, the pulse frequency of the high-frequency environment provided by the measurement circuit 610 equals to that of the electrical stimulation signal.

According to an embodiment of the present invention, the impedance compensation device 600 may be equipped within the external control device 200. According to another embodiment of the present invention, the impedance compensation device 600 may be equipped within the electrical stimulation device 100. That is, the high-frequency environment may be provided by the electrical stimulation device 100 or the external control device 200. In addition, According to another embodiment of the present invention, the impedance compensation device 600 may be an independent device (e.g. an impedance analyzer).

According to an embodiment of the present invention, the impedance compensation device 600 may apply to the trial phase (i.e. the electrical stimulation device 100 is an external electrical stimulation device with a lead implanted into the human body). According to an embodiment of the present invention, the impedance compensation device 600 may apply to the permanent implantation phase (i.e. the electrical stimulation device 100 is an implantable electrical stimulation device, and the electrical stimulation device 100 may be implanted into the human body along with the lead).

According to an embodiment of the present invention, the impedance compensation device 600 may apply to the electrical stimulation device 100 before leaving the factory (e.g. in a laboratory or factory). In an embodiment, before the electrical stimulation device 100 leaves the factory, the impedance compensation device 600 may first calculate the impedance Z_{Lead} of the lead and the impedance Z_{Inner} of the electrical stimulation device 100, and writes the calculated impedance Z_{Lead} of the lead and the impedance Z_{Inner} of the electrical stimulation device 100 into the firmware of the electrical stimulation device 100. In another embodiment, before the electrical stimulation device 100 leaves the factory, the impedance compensation device 600 may first calculate the impedance Z_{Inner} of the electrical stimulation device 100, and writes the calculated impedance Z_{Inner} of the electrical stimulation device 100 into the firmware of the electrical stimulation device 100. According to an embodiment of the present invention, in the electrical stimulation phase and the non-electrical stimulation phase, the impedance compensation device 600 may perform real-time compensation; that is, each time an electrical stimulation signal is sent, the impedances Z_{Inner} and Z_{Lead} may be measured.

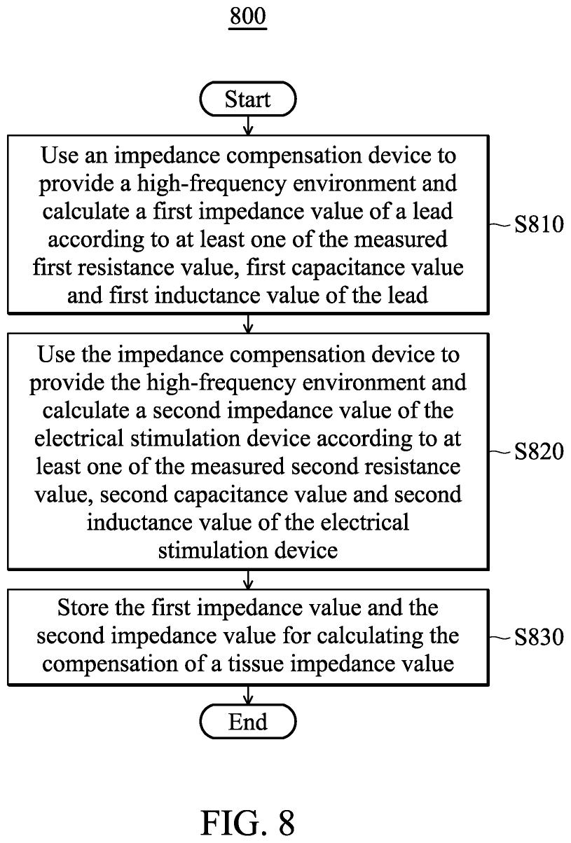

FIG. 8 shows a flow diagram 800 of an electrical stimulation method for impedance compensation according to an embodiment of the present invention. The flow diagram 800 of an electrical stimulation method for impedance compensation applies to the electrical stimulation device 100 and the impedance compensation device 600 that provide high-frequency electrical stimulation. As shown in FIG. 8, in step S810, the impedance compensation device 600 provides a high-frequency environment, and calculates a first impedance value of a lead according to at least one of the measured first resistance value, the measured first capacitance value, or the measured first inductance value of the lead.

In step S820, the impedance compensation device 600 provides the high-frequency environment, and calculates a second impedance value of the electrical stimulation device 100 according to at least one of the measured second resistance value, the measured second capacitance value and the measured second inductance value of the electrical stimulation device 100.

In step S830, the electrical stimulation device 100 stores the first impedance value and the second impedance value for calculating the compensation for the tissue impedance value.

According to an embodiment of the present invention, in the aforementioned electrical stimulation method for impedance compensation, the electrical stimulation device 100 may calculate the total impedance value, and obtain the tissue impedance by subtracting the first impedance value of the lead and the second impedance value of the electrical stimulation device 100 from the total impedance value.

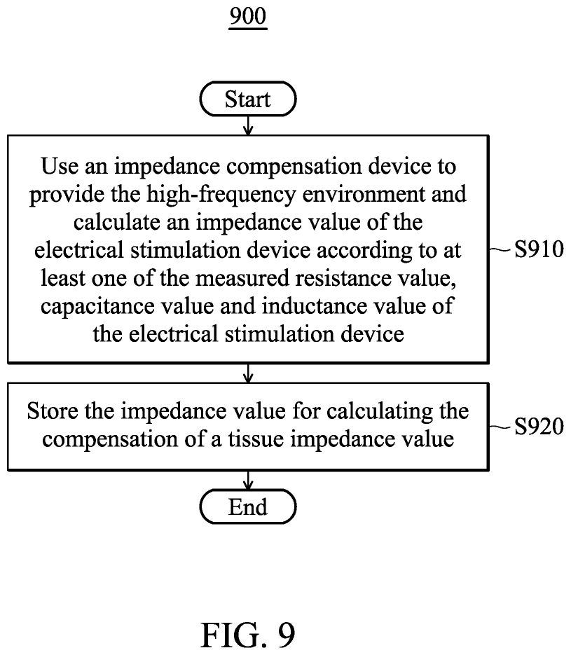

FIG. 9 shows a flow diagram 900 of an electrical stimulation method for impedance compensation according to another embodiment of the present invention. The flow diagram 900 of an electrical stimulation method for impedance compensation applies to the electrical stimulation device 100 and the impedance compensation device 600 that provide a high-frequency electrical stimulation. As shown in FIG. 9, in step S910, the impedance compensation device 600 provides a high-frequency environment, and calculates an impedance value of the electrical stimulation device 100 according to at least one of the measured resistance value, capacitance value and inductance value of the electrical stimulation device 100.

In step S920, the electrical stimulation device 100 stores the impedance value for calculating the compensation for the tissue impedance value.

According to an embodiment of the present invention, in the aforementioned electrical stimulation method for impedance compensation, the electrical stimulation device 100 may calculate the total impedance value, and obtain the tissue impedance by subtracting the impedance value of the electrical stimulation device 100 from the total impedance value.

According to the electrical stimulation method for impedance compensation provided by the present invention, when the electrical stimulation device is calculating the tissue impedance value, it may refer to pre-calculated lead impedance and an electrical stimulation device impedance to calculate the tissue impedance, so as to compensate for a possible error when calculating the tissue impedance. Thus, according to the electrical stimulation method for impedance compensation provided by the present invention, the electrical stimulation device may obtain a more accurate tissue impedance value.

The ordinal numbers used herein and in the claims, such as “first,” “second” and the like, are used merely for the convenience of description and have no sequential orders among each other.

The steps of the methods and algorithms disclosed herein may be directly applied to hardware, a software module or the combination thereof by executing a processor. A software module (including execution commands and related data) and other data may be stored in a data memory, such as a random access memory (RAM), a flash memory, a read-only memory (ROM), an erasable programmable read-only memory (EPROM), a register, a hard disk drive, a portable hard disk drive, a compact disc read-only memory (CD-ROM), a digital video/versatile disc (DVD), or any other computer-readable storage media format well-known in the art. A storage media may be coupled to a machine device, such as a computer/processor (indicated as a processor herein for the convenience of description), wherein the processor may read information (e.g. a program code) and write information into a storage media. A storage media may be integrated with a processor. An application specific integrated circuit (ASIC) includes a processor and a storage media. A user apparatus includes an ASIC. In other words, a processor and a storage media are included in a user apparatus in a manner that are not directly connected to the user apparatus. In addition, in some embodiments, any product suitable for a computer program includes a readable storage media, wherein a readable storage media includes a program code related to one or more embodiments disclosed herein. In some embodiments, a product of a computer program may include a packaging material.

Various aspects are described in the above description. The teaching herein may be implemented in various ways, and any particular structure or function disclosed in the examples is merely a representative case. According to the teaching herein, it is understood by persons skilled in the art that each aspect of the disclosure herein may be implemented independently, or alternatively, two or more aspects may be implemented in combination.

While the present disclosure has been described in terms of the embodiments, it should be understood that the embodiments are not intended to limit the present disclosure therein. Persons skilled in the art may perform modifications and alterations without departing from the spirit and scope of the present disclosure. Thus, the scope of the invention should be determined by the appended claims.

What is claimed is:

1. An electrical stimulation method for impedance compensation applied to an electrical stimulation device that provides a high-frequency electrical stimulation, the method comprising:
by an impedance compensation device, providing a high-frequency environment and calculating a first impedance value of a lead according to at least one of a measured first resistance value, a measured first capacitance value and a measured first inductance value of the lead;
by the impedance compensation device, providing the high-frequency environment and calculating a second impedance value of the electrical stimulation device according to at least one of a measured second resistance value, a measured second capacitance value, or a measured second inductance value of the electrical stimulation device; and
storing the first impedance value and the second impedance value for calculating a tissue impedance value.

2. The electrical stimulation method for impedance compensation as claimed in claim 1, wherein the high-frequency environment is simulated with an electrical stimulation frequency used by the electrical stimulation device.

3. The electrical stimulation method for impedance compensation as claimed in claim 1, wherein a pulse frequency range of the high-frequency environment is between 1 KHz and 1000 KHz.

4. The electrical stimulation method for impedance compensation as claimed in claim 1, wherein the impedance compensation device is an external device or is arranged within the electrical stimulation device.

5. The electrical stimulation method for impedance compensation as claimed in claim 1, wherein the lead is implanted into a tissue of a human body.

6. The electrical stimulation method for impedance compensation as claimed in claim 1, wherein the lead and the electrical stimulation device are implanted into a tissue of a human body.

7. An electrical stimulation system, applicable to a high-frequency electrical stimulation operation, the system including:
a lead;
an electrical stimulation device; and
an impedance compensation device, providing a high-frequency environment, calculating a first impedance value of the lead according to at least one of a measured first resistance value, a measured first capacitance value and a measured first inductance value of the lead, and calculating a second impedance value of the electrical stimulation device according to at least one of a measured second resistance value, a measured second capacitance value, or a measured second inductance value of the electrical stimulation device;
wherein the first impedance value and the second impedance value are stored in the electrical stimulation device for calculating a compensation for a tissue impedance value.

8. The electrical stimulation system as claimed in claim 7, wherein the impedance compensation device simulates the high-frequency environment with an electrical stimulation frequency used by the electrical stimulation device.

9. The electrical stimulation system as claimed in claim 7, wherein a pulse frequency range of the high-frequency environment is between 1 KHz and 1000 KHz.

10. The electrical stimulation system as claimed in claim 7, wherein the impedance compensation device is an external device or is arranged within the electrical stimulation device.

11. The electrical stimulation system as claimed in claim 7, wherein the lead is implanted into a tissue of a human body.

12. The electrical stimulation system as claimed in claim 7, wherein the lead and the electrical stimulation device are implanted into a tissue of a human body.

13. An electrical stimulation method for impedance compensation applied to an electrical stimulation device that provides a high-frequency electrical stimulation, the method comprising:
by an impedance compensation device, providing a high-frequency environment and calculating an impedance value of a lead according to at least one of a measured resistance value, a measured capacitance value, or a measured inductance value of the electrical stimulation device; and
storing the impedance value for calculating a tissue impedance value.

14. The electrical stimulation method for impedance compensation as claimed in claim 13, wherein the high-frequency environment is simulated with an electrical stimulation frequency used by the electrical stimulation device.

15. The electrical stimulation method for impedance compensation as claimed in claim 13, wherein a pulse frequency range of the high-frequency environment is between 1 KHz and 1000 KHz.

16. The electrical stimulation method for impedance compensation as claimed in claim 13, wherein the impedance compensation device is an external device or is arranged within the electrical stimulation device.

17. An electrical stimulation system, applicable to a high-frequency electrical stimulation operation, the system including:
an electrical stimulation device; and
an impedance compensation device, providing a high-frequency environment, calculating an impedance value of the electrical stimulation device according to at least one of a measured resistance value, a measured capacitance value, or a measured inductance value of the electrical stimulation device;
wherein the impedance value is stored in the electrical stimulation device for calculating a tissue impedance value.

18. The electrical stimulation system as claimed in claim 17, wherein the impedance compensation device simulates the high-frequency environment with an electrical stimulation frequency used by the electrical stimulation device.

19. The electrical stimulation system as claimed in claim 17, wherein a pulse frequency range of the high-frequency environment is between 1 KHz and 1000 KHz.

20. The electrical stimulation system as claimed in claim 17, wherein the impedance compensation device is an external device or is arranged within the electrical stimulation device.
